(19) United States
(12) Patent Application Publication
(10) Pub. No.: US 2016/0095532 A1
(43) Pub. Date: Apr. 7, 2016

(54) METHOD AND COMPONENTS FOR IN VIVO DETERMINATION OF MALIGNANCY

(71) Applicant: WEINBERG MEDICAL PHYSICS LLC, Bethesda, MD (US)

(72) Inventors: Irving N. WEINBERG, Bethesda, MD (US); Wolfgang LOSERT, College Park, MD (US)

(21) Appl. No.: 14/874,804

(22) Filed: Oct. 5, 2015

Related U.S. Application Data
(60) Provisional application No. 62/059,220, filed on Oct. 3, 2014.

(51) Int. Cl. A61B 5/055; A61B 5/107; A61B 5/08; A61B 5/00

(52) U.S. Cl. CPC A61B 5/055; A61B 5/7275; A61B 5/1075; A61B 5/4312; A61B 5/4381; A61B 5/08; A61B 5/7207; A61B 5/7246

(57) ABSTRACT

An apparatus and method apply magnetic fields by generators external to a body or body part with sensors within an in vivo source that are sensitive to applied magnetic fields Through the use of these applied magnetic fields and sensitive sensors, disclosed embodiments can realize much better spatial resolution than is conventionally possible.

### CROSS REFERENCE

This application claims priority under 35 U.S.C. 119(e) to U.S. Provisional Patent Application 62/059,220 (incorporated by reference in its entirety) on Oct. 3, 2014, entitled “NEW GOLD STANDARD FOR IN VIVO DETERMINATION OF MALIGNANCY.”

### FIELD OF THE INVENTION

Disclosed embodiments are directed to high-resolution functional imaging in a body, or body part, in particular using magnetic imaging and particle imaging instruments.

### SUMMARY

Disclosed embodiments provide the ability to apply very high resolution magnetic resonance images (“MRI) (for example with spatial resolution of better than 25 microns full-width at half-maximum) over a short time period (for example, one hour) to tissue in situ, in order to detect abnormally directed or shaped unicellular or multicellular motion.

In accordance with at least one disclosed embodiment, such abnormal motion may be used to characterize a portion of the tissue in terms of the likelihood of malignancy within that tissue, or the growth rate of that tissue.

In accordance with at least one disclosed embodiment, characterization of cells may be accomplished also through the application of magnetic polarizing pulses that may be spatially and temporally selective, so as to assess the internal dynamics of cells (e.g., actin formation) or groups of cells.

In accordance with at least one disclosed embodiment, potential confounding factors such as motion un-sharpness due to gross movement of the body part may be reduced or eliminated with the use of immobilization techniques (e.g., breast compression), or image-tracking or motion-correction algorithms.

### BRIEF DESCRIPTION OF THE FIGURES

The detailed description particularly refers to the accompanying figures in which:

FIG. 1 illustrates an envisaged example of a field-of-view of cells within two consecutive images collected with disclosed embodiments of a high resolution MRI system.

FIG. 2 illustrates an envisaged example of a field-of-view of cells within two consecutive images collected with disclosed embodiments of a high resolution MRI system.

FIG. 3 illustrates an embodiment of a high resolution MRI system for performing the disclosed functionality and methodologies.

### DETAILED DESCRIPTION

For centuries, pathologists have been called upon to examine tumor specimens in order to determine whether the tumor is benign or malignant. This privileged role is a result of the fact that it has not been heretofore possible to finely and confidently examine tissues while they are still inside the patient. As a result, some or all of the tumor is removed from the body by a medical practitioner, and the pathologist is asked to examine a small segment of that portion using gross and histological inspection. The latter consists of examination of thin samples after the specimen has been stained with dyes selective for various structures.

The above traditional approach for determination of malignancy is wanting in several respects. For example, many patients would prefer not to have their bodies punctured in order to remove tissue samples. Likewise, pathologists only examine a small portion of the tissue removed. Rarely, the act of removing a portion of a malignant tumor causes the tumor cells to be spread. Finally, the examination process only provides indirect measures of malignancy, rather than basic properties such as metastatic activity or growth rate.

Conventional medicine also subscribes to the idea that a robust method of assessing the malignancy of a tumor is to examine its growth over time. This principle is used in scheduling colonoscopies as well as lung CT scans. However, as described in the scientific paper by Michael Poullis et al., entitled “Biology of colorectal pulmonary metastasis: implications for surgical resection” and published in the Journal Interactive CardioVascular and Thoracic Surgery (2012, volume 14, pages 140-142) (incorporated herein by reference), the doubling time for a sub-centimeter (i.e., 5 mm) tumor is between 30 and 120 days. This measurement implies that the edge of the tumor travels an average of 2 mm in as long a period of 120 days, or an average of about one micron per hour.

It is also conventionally known that cells may migrate from a tumor into adjacent normal tissue, which is believed to lead to cause therapeutic failures, as taught by the scientific article authored by Daniel L. Silbergeld and Michael R. Chicoine, entitled “Isolation and characterization of human malignant cells from histologically normal brain,” published in the Journal of Neurosurgery in 1997 (volume 86, pages 525-531) (incorporated herein by reference). In that article, the authors quoted a migration velocity of 12 microns per hour.

It is further known that cells exhibit motion during such migration, and that patterns of this motion can be discerned with rapid imaging methods, as described in the publication by Chenlu Wang et al entitled “The interplay of cell-cell and cell-substrate adhesion in collective cell migration,” published online in the 2014 Journal of the Royal Society Interface (volume 11, number 100) (incorporated herein by reference).

With this understanding of the conventional tumor investigation and imaging in mind, disclosed embodiments provide the ability to apply very high resolution magnetic resonance images (“MRI) (for example with spatial resolution of 25 microns full-width at half-maximum) over a short time period (for example, one hour) to tissue in situ, in order to detect abnormally directed or shaped unicellular or multicellular motion. Such abnormal motion can be used to characterize a portion of the tissue in terms of the likelihood of malignancy within that tissue, or the growth rate of that tissue. Thus, disclosed embodiments provide a novel method of detecting and characterizing the malignancy of a tissue in the body.

The presently disclosed embodiments are based in part on prior inventions by one of the present inventors, Dr. Irving Weinberg, which evidence that it is possible to impose magnetic gradients on a subject, wherein the magnetic gradients have very high magnitudes without discomfort to the subject if the rise- and fall-times of the imposition were less than conventionally used (i.e., less than 10 microseconds). These inventions include those disclosed and claimed in U.S. Pat. Nos. 8,466,680 and 8,154,286, and related filed patent applications cross referenced and/or related (by priority claim) to those patents (each of which being incorporated by reference).

Presently disclosed embodiments utilize such fast and strong magnetic fields to obtain images rapidly, and with very good spatial resolution that is on the same order of size as single cells.

Further, one of the present inventors, Dr. Irving Weinberg, developed a method and components for achieving imaging spatial resolution of 20 microns with MRI, as described in the Proceedings of the Annual 2014 Meeting of the ISMRM in a poster entitled “A quiet, fast, high-resolution desktop MRI capable of imaging solids,” (incorporated herein by reference).

It is well-known that the imaging characteristics of a lesion's edge may provide clues as to whether the lesion is benign or malignant. For example, the ultrasound determination of indistinct tumor margins is suggestive of malignancy, as described in the 2006 article entitled “Characterization of Solid Breast Masses,” by M Constantini et al., published in the Journal of Ultrasound in Medicine (volume 25, pages 649-659) (incorporated herein by reference). One of the inventors, Wolfgang Losert, has shown that inspection of the edge of a cell and of the edge and internal dynamics of a group of cells can provide information as to the local chemical environment, as described in the 2012 article entitled “Cell Shape Dynamics: From Waves to Migration”, by M. K. Driscoll et al., in the journal PLOS Computation Biology (volume 8, number 3) (incorporated herein by reference).

Disclosed embodiments enable inspection of the edge of a lesion with high spatial resolution within two or more images so as to provide a description of the motion of the cells at the edge of the lesion, and hence add to diagnostic information about the lesion. The images in the publication by M. K. Driscoll were collected optically, which would be possible for a superficial lesion but not for a lesion deep in the body.

To the contrary, the disclosed embodiments provide the ability to examined a lesion within the body, in vivo, in such a manner that magnetic resonance imaging may be performed with, for example, a spatial resolution of 25 microns full-width at half-maximum, over a short time period (for example, one hour) to tissue in situ, in order to detect abnormally directed or shaped unicellular or multicellular motion. In this way, the disclosed embodiments provide a novel and unobvious analytic technique that may be utilized by practitioners prior to and as a basis for determining whether to biopsy a tissue. In other words, one of the bases for determining whether to biopsy a tissue may be the identification of suspicious cellular motion using the disclosed embodiments.

As a result of obtaining and inspecting images of tissue in vivo with high spatial resolution, e.g., better than 25 microns full-width at half-maximum, sufficient information is obtained to analyze an edge of a lesion (as performed by M. K. Driscoll for cell cultures) and derive information about the malignancy or malignant potential of the lesion.

Moreover, a high spatial resolution MRI instrument may be used to characterize cells within still-living tissue after its removal from the body in order to ascertain the presence of and/or characterize malignancy in that tissue.

For the purpose of this specification, microscopic cellular characteristics is defined as the collection of data about the shape, orientation, and motion of one or more cells at microscopic resolution, defined as better than 25 microns full-width at half-maximum.

FIG. 1 illustrates an envisaged example of a field-of-view of cells within two consecutive images 100, 110, collected with a high resolution MRI system designed in accordance with the disclosed embodiments. In the second image 110 illustrated in FIG. 1, a cell 120 has moved, thereby suggesting the presence of malignancy.

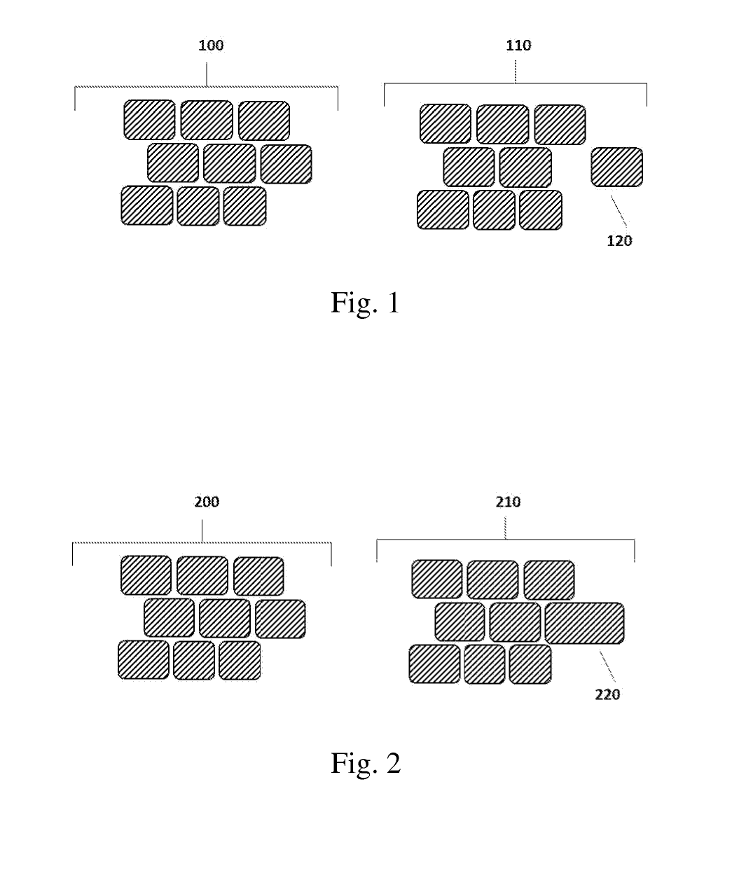

FIG. 2 similarly illustrates an envisaged example of a field-of-view of cells within two consecutive images 200, 210 collected with a high resolution MRI system designed in accordance with the disclosed embodiments. In the second image 210 of FIG. 2, a cell 220 has changed its appearance, thereby suggesting the presence of malignancy.

Inspection of multiple, high resolution images provides more extensive detail for analysis of the edge of a cell or edge of groups of cells, for example, cells at the edge of the lesion. More specifically, by obtaining at least two images, data are available regarding the position and condition of the edge of a cell (or group of cells) at time t1, the position and condition of the edge of a cell (or group of cells) at time t2, and the alteration in position and condition occurring between t1 and t2. Furthermore, inspection of multiple, high resolution images provides more extensive detail for analysis of internal dynamics within a cell or group of cells.

As a result, of the disclosed embodiments, characterization of cells can be accomplished also through the application of magnetic polarizing pulses that could be spatially and temporally selective, so as to assess the internal dynamics of cells (e.g., actin wave-like dynamics) or of groups of cells. See, M. K. Driscoll et al. incorporated herein in its entirety. For example, a diffusion-weighted MR imaging could yield a different signal for orientation of the actin fibers in one direction than in another direction.

Potential confounding factors such as motion un-sharpness due to gross movement of the body part may be reduced or eliminated with the use of immobilization techniques (e.g., breast compression), or image-tracking or motion-correction algorithms. Such motion-correction algorithms may correct for body part position changes between images. Body part position changes may be in the form of movement of the body part being imaged or translation, squeezing/expansion of the body part.

It should be understood that the gradient-generating coils are under the control of a controller that enables, automatic, semi-automatic and/or manual control of generated magnetic fields and magnetic gradients.

It is understood that the term “radiation” includes emission and reflection of RF energy, and also includes other methods of transmitting information over a distance, for example with entangled quantum effects.

It should be understood that control and cooperation of the components of the instrument may be provided using software instructions that may be stored in a tangible, non-transitory storage device such as a non-transitory computer readable storage device storing instructions which, when executed on one or more programmed processors, carry out the above-described method operations and resulting functionality. In this case, the term non-transitory is intended to preclude transmitted signals and propagating waves, but not storage devices that are erasable or dependent upon power sources to retain information.

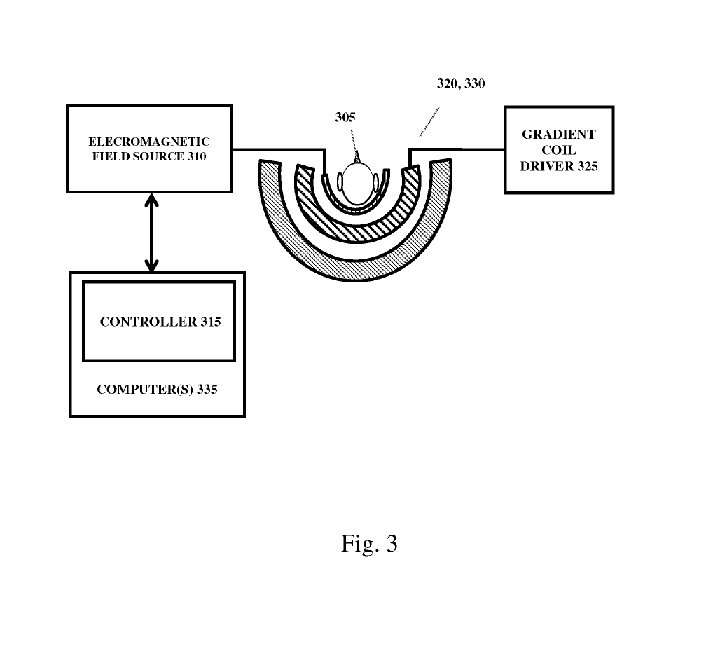

FIG. 3 illustrates an apparatus for performing the disclosed functionality. Such an apparatus 300 includes a controllable electromagnetic field source 310, which includes a controller 315 that enables control of a static magnetic field. The apparatus 300 also includes a gradient coil or coils 320 (which is also under control of the controller 315 to enables control of the gradient to produce a magnetic gradient in a volume-of-interest encompassing some or all of a tissue sample of a subject 305 using at least one coil driver 325. The apparatus 310 further comprises a coil or coils 330 for transmitting and/or receiving RF energy into and from the tissue sample of the body part so as to perform in vivo magnetic resonance imaging to evaluate cellular dynamics in a body part to provide diagnostic, prognostic, and treatment planning information.

It should be understood that control and cooperation of the components of the apparatus 300 may be provided using software instructions that may be stored in a tangible, non-transitory storage device such as a non-transitory computer readable storage device storing instructions which, when executed on one or more programmed processors, carry out a method of imaging the volume of interest within the sample. In this case, the term non-transitory is intended to preclude transmitted signals and propagating waves, but not storage devices that are erasable or dependent upon power sources to retain information.

It should be understood that the components illustrated in FIG. 3 and their associated functionality may be implemented in conjunction with, or under the control of, one or more general purpose computers 335 running software algorithms to provide the presently disclosed functionality, in particular, analysis of imaging data for detection of movement for characterization of tissue. As a result, such software turns those computers into specific purpose computers.

It should be understood that the disclosed embodiments also encompass a method of operating the disclosed apparatus wherein a magnet field source and at least one coil under control of a control unit are used to establish a magnetic field and magnetic gradient, radio frequency energy is generated and transmitted into and received from the sample under the control of the control unit to obtain imaging data regarding the sample such that very high resolution MRI (for example with spatial resolution of better than 25 microns full-width at half-maximum) may be generated over a short time period (for example, one hour) to tissue in situ, in order to detect abnormally directed or shaped unicellular or multicellular motion.

In accordance with at least one disclosed embodiment, such abnormal motion may be used to characterize a portion of the tissue in terms of the likelihood of malignancy within that tissue, or the growth rate of that tissue.

In accordance with at least one disclosed embodiment, characterization of cells may be accomplished also through the application of magnetic polarizing pulses that may be spatially and temporally selective, so as to assess the internal dynamics of cells (e.g., actin wave-like dynamics) or of groups of cells.

It should be understood that the components illustrated in FIG. 3 and their associated functionality may be implemented in conjunction with, or under the control of, one or more general purpose computers running software algorithms to provide the presently disclosed functionality and turning those computers into specific purpose computers.

Moreover, those skilled in the art will recognize, upon consideration of the above teachings, that the above exemplary embodiments may be based upon use of one or more programmed processors programmed with a suitable computer program. However, the disclosed embodiments could be implemented using hardware component equivalents such as special purpose hardware and/or dedicated processors. Similarly, general purpose computers, microprocessor based computers, micro-controllers, optical computers, analog computers, dedicated processors, application specific circuits and/or dedicated hard wired logic may be used to construct alternative equivalent embodiments.

Those skilled in the art will appreciate, upon consideration of the above teachings, that the program operations and processes and associated data used to implement certain of the embodiments described above can be implemented using disc storage as well as other forms of storage devices including, but not limited to non-transitory storage media (where non-transitory is intended only to preclude propagating signals and not signals which are transitory in that they are erased by removal of power or explicit acts of erasure) such as for example Read Only Memory (ROM) devices, Random Access Memory (RAM) devices, network memory devices, optical storage elements, magnetic storage elements, magneto-optical storage elements, flash memory, core memory and/or other equivalent volatile and non-volatile storage technologies without departing from certain embodiments of the present invention. Such alternative storage devices should be considered equivalents.

While certain illustrative embodiments have been described, it is evident that many alternatives, modifications, permutations and variations will become apparent to those skilled in the art in light of the foregoing description. While illustrated embodiments have been outlined above, it is evident that many alternatives, modifications and variations will be apparent to those skilled in the art. Accordingly, the various embodiments of the invention, as set forth above, are intended to be illustrative, not limiting. Various changes may be made without departing from the spirit and scope of the invention.

As a result, it will be apparent for those skilled in the art that the illustrative embodiments described are only examples and that various modifications can be made within the scope of the invention as defined in the appended claims.

We claim:

1. A method of using in vivo magnetic resonance imaging to evaluate the microscopic cellular characteristics of one or more cells in a body part in order to provide diagnostic, prognostic, and treatment planning information, the method comprising:
generating at least one magnetic gradient in the body part using at least one coil under control of a control unit; and
detecting radio frequency energy received from the body part to obtain imaging data regarding the body part so as to perform magnetic resonance imaging of the body part to detect the shape and motion of cells in the body part; and
predicting the malignancy and/or malignancy-potential of tissues in the body part based on the cellular characteristics,
wherein the spatial resolution of the magnetic resonance imaging instrument is better than 25 microns full-width at half-maximum.

2. The method of claim 1, wherein the detected cellular characteristics include cellular motion within the tissues in the body part under magnetic resonance imaging.

3. The method of claim 1, wherein the detected cellular characteristics include the shape of one or more cells within the tissues in the body part under magnetic resonance imaging.

4. The method of claim 1, wherein the MRI examination of the cells in the tissues of the body part is performed after removal of the tissues from the body part.

5. The method of claim 1, wherein detection of the microscopic cellular characteristics is performed by obtaining imaging data regarding the body part at least two times.

6. The method of claim 1, wherein the body part is a human breast.

7. The method of claim 1, wherein the body part is a human prostate.

8. The method of claim 1, wherein the body part is a human lung.

9. The method of claim 1, wherein motion-unsharpness is reduced or eliminated through compression and/or image motion correction.

10. The method of claim 1, wherein the magnetic resonance imaging employs magnetic gradients with field strength greater than 0.1 Tesla/meter.

11. The method of claim 1, wherein the magnetic resonance imaging does not cause bio-effects within the tissues being imaged.

12. The method of claim 1, wherein the gradient field rises in less than 10 microseconds.

13. The method of claim 1, where the tissues are characterized by collecting at least two MR images of the body part and observing changes in the cellular characteristics between the at least two images.

14. The method of claim 13, wherein the at least two magnetic resonance images are obtained within a period of one week or less.

15. The method of claim 1, wherein the tissues are characterized by examining microscopic patterns of the tumor edge in magnetic resonance images of the body part.

16. The method of claim 1, wherein the tissues are characterized by examining differences in microscopic patterns of the tumor edge in at least two MR images of the body part.

17. A magnetic resonance imaging apparatus using in vivo magnetic resonance imaging to evaluate cellular dynamics in a body part to provide diagnostic, prognostic, and treatment planning information, the apparatus comprising:
at least one coil; and
a control unit,
wherein the at least one coil is under the control of the control unit to generate and transmit radio frequency energy into the body part,
wherein radio frequency energy is received from the body part and analyzed to obtain imaging data regarding the body part so as to perform magnetic resonance imaging of the body part to detect cellular characteristics,
wherein malignancy and/or malignancy-potential of tissues in the body part are characterized based on the detected cellular characteristics, and
wherein a spatial resolution of the magnetic resonance imaging apparatus is better than 25 microns.

18. The apparatus of claim 17, wherein the detected cellular characteristics include the shape of one or more cells within the tissues in the body part under magnetic resonance imaging.

19. The apparatus of claim 17, wherein the MRI examination of the cells in the tissues of the body part is performed after removal of the tissues from the body part.

20. The apparatus of claim 17, wherein the detected cellular characteristics include cellular motion within the tissues in the body part under magnetic resonance imaging.

21. The apparatus of claim 20, wherein detection of the cellular motion is performed by obtaining imaging data regarding the body part at at least two times.

22. The apparatus of claim 17, further comprising at least one coil driver coupled to the at least one coil and driving the at least one coil to generate a magnetic field gradient under the control of the control unit.

23. The apparatus of claim 17, wherein the body part is a human breast.

24. The apparatus of claim 17, wherein the body part is a human prostate.

25. The apparatus of claim 17, wherein the body part is a human lung.

26. The apparatus of claim 17, wherein motion-unsharpness is reduced or eliminated through compression and/or image motion correction.

27. The apparatus of claim 17, wherein the magnetic resonance imaging employs magnetic gradients with field strength greater than 0.1 Tesla/meter.

28. The apparatus of claim 17, wherein the magnetic resonance imaging does not cause bio-effects within the tissues being imaged.

29. The apparatus of claim 17, wherein the gradient field rises in less than 10 microseconds.

30. The apparatus of claim 17, where the tissues are characterized by collecting at least two MR images of the body part and observing changes in cellular configuration between the at least two images.

31. The apparatus of claim 30, wherein the at least two magnetic resonance images are obtained within a period of one week or less.

32. The apparatus of claim 17, wherein the tissues are characterized by examining microscopic patterns of the tumor edge in magnetic resonance images of the body part.

33. The apparatus of claim 17, wherein the tissues are characterized by examining differences in microscopic patterns of the tumor edge in at least two MR images of the body part.
